(19) United States
(12) Patent Application Publication
(10) Pub. No.: US 2020/0276288 A1
(43) Pub. Date: Sep. 3, 2020

(54) METHOD OF MAKING A PERSONALIZED CANCER VACCINE

(71) Applicant: FLOW PHARMA, INC., Redwood City, CA (US)

(72) Inventors: REID M. RUBSAMEN, Alamo, CA (US); CHARLES V. HERST, Oakland, CA (US); LU WANG, San Jose, CA (US); SCOTT R. BURKHOLZ, Greer, SC (US); RICHARD T. CARBACK, III, Everett, MA (US); SERBAN I. CIOTOS, San Jose, CA (US)

(73) Assignee: FLOW PHARMA, INC., Redwood City, CA (US)

(21) Appl. No.: 16/806,664

(22) Filed: Mar. 2, 2020

Related U.S. Application Data
(60) Provisional application No. 62/812,723, filed on Mar. 1, 2019.

(51) Int. Cl. A61K 39/00; G16B 40/00; G06F 17/18; G16B 5/00

(52) U.S. Cl. CPC A61K 39/0011; G16B 5/00; G06F 17/18; G16B 40/00

(57) ABSTRACT

A method of making a personalized cancer vaccine is disclosed. The method includes predicting whether a first neoantigen or a second neoantigen of an individual cancer patient has a stronger binding affinity for a human leukocyte antigen (HLA) complex of the patient and creating a particle containing the neoantigen with the stronger predicted binding affinity. The predicting step can be implemented using artificial intelligence, statistical modeling, or a combination thereof. Particles are created by encapsulating the neoantigen with the stronger predicted binding affinity for the HLA complex of the patient in a biocompatible material. Placing the antigen in a particular sized particle is referred to here as Size Exclusion Antigen Presentation Control, (SEAPAC) used in methods of treating the patient using such a personalized cancer vaccine.

### FIELD OF THE INVENTION

This invention relates generally to the field of personalized cancer vaccines. The invention relates to the design of personalized cancer vaccines, e.g. the selection of which neoantigen(s) to include in a personalized cancer vaccine, along with the manufacture and use of such vaccines.

### BACKGROUND OF THE INVENTION

The term vaccine derives from Edward Jenner's 1796 use of the term cow pox (Latin variolævaccinæ, adapted from the Latin vaccīn-us, from vacca cow), which, when administered to humans, provided them protection against smallpox. The 20th century saw the introduction of several successful vaccines for communicable diseases, such as those for diphtheria, measles, mumps, and rubella.

However, cancer, a non-communicable disease, also causes enormous burdens on society. In fact, the world population experienced an estimated 18.1 million new cases of cancer in 2018. Traditionally, cancer treatment involved chemical or biological compounds (chemotherapy), radiation (radiotherapy), or surgery. However, additional anti-cancer countermeasures have been developed in recent years, including immunotherapy treatments such as personalized cancer vaccines.

A personalized cancer vaccine helps fight cancer by exposing the patient to one or more neoantigens, which are antigens present on the surface of cancer cells but absent from the surface of normal cells. After a neoantigen is administered to the patient, an antigen presenting cell (APC) of the patient's immune system ingests the neoantigen. The neoantigen undergoes intracellular processes in the APC where it is digested, transported, and subsequently bound to present on the surface of the cell as a complex with a human leukocyte antigen (HLA). Other immune cells, such as cytotoxic T cells, can then recognize the HLA-neoantigen complex on the surface of the APC, prompting them to attack cells that display the neoantigen, such as cancer cells. Hence, a personalized cancer vaccine helps a patient's immune system to identify, and thereby kill, cancer cells.

To date, however, the clinical efficacy of personalized cancer vaccines has been less than desired. Cancerous tumors continue to grow and spread in many patients despite treatment with personalized cancer vaccines.

To address low vaccine efficacy, it has been hypothesized that not all neoantigens are equally able to stimulate an immune response. In particular, some neoantigens have only weak binding affinity for a patient's HLA complex, and therefore no HLA-neoantigen complex will form. Without such an HLA-neoantigen complex, no immune response will be generated based on that neoantigen. In fact, it has been estimated that only about 0.5% to 1% of neoantigens bind sufficiently to an HLA complex to induce a sufficient immune response (Yewdell et al, Annu Rev Immunol., 199917, 51-88). Therefore, predicting which neoantigens will effectively bind to a patient's HLA complex could lead to more effective personalized cancer vaccines.

If a single APC ingests and then simultaneously attempts to present more than one neoantigen to a T-Cell, the two neoantigens could competitively inhibit at the motif and result in only one neoantigen being presented. Even if this problem is overcome, successful presentation of large numbers on neoantigens can result in immunodominance, a phenomenon where only a subset of the successfully presented neoantigens result in T-Cell attack on cancer cells. Methods for relieving the effects of immunodominance are described in published US patent application 2008/0260780, entitled “Materials And Methods Relating To Improved Vaccination Strategies”; US patent application 2009/0269362, entitled “Method for Controlling Immunodominance”; and US patent application 2010/0119535, entitled “Compositions and Methods for Immunodominant Antigens” incorporated here by reference to disclose and describe such methods.

### SUMMARY OF THE INVENTION

A patient personalized cancer vaccine is disclosed. The vaccine is comprised of the biocompatible material which may be a polymer formed into a spherical particle having a diameter of 10 micrometers to 25 micrometers ±5%, 10%, 20%. Inside the particles is a first neoantigen which is predicted to have a stronger binding affinity for an HLA complex of a patient as compared to an HLA binding affinity of a second neoantigen.

The vaccine is produced by first identifying first and second neoantigens. Second, the human leukocyte antigen (HLA) genotype of the patient is determined. Next, a prediction is made on whether the first neoantigen or the second neoantigen has a stronger binding affinity for an HLA complex of the patient being treated based on training data of the HLA genotype of the patient. The vaccine is created by placing particles of the neoantigens inside of the spherical polymer particles and creating an injectable formulation for administration to the patient.

The personalized vaccine may be for a cancer patient such as a triple negative breast cancer tumor that does not produce programmed death-ligand 1 (PD-L1) above a level of 1.5 fragments per kilobase per million mapped reads or a level above 2 fragments, 5 fragments, or 10 fragments per kilobase per million mapped reads. The particles are designed so as to have a diameter such that only a single particle can be consumed by an antigen presenting cell such as a dendritic cell, and as such, may have a diameter of about 11 microns ±20%, ±10%, ±5% when used in connection with a human patient.

Methods and compositions related to personalized cancer vaccines are disclosed. The disclosure provides methods of making a personalized cancer vaccine that includes predicting whether a first neoantigen or a second neoantigen of a particular individual patient has a stronger binding affinity for a human leukocyte antigen (HLA) complex of the patient and creating a particle containing the neoantigen with the stronger predicted binding affinity. Such a predicting step includes artificial intelligence, statistical modeling, or a combination thereof. Such a particle is created by encapsulating the neoantigen with the stronger predicted binding affinity for the HLA complex of the patient in a material. Placing the antigen in a particular sized particle is referred to here as Size Exclusion Antigen Presentation Control, (SEAPAC). The disclosure also provides methods of treating the patient using such a personalized cancer vaccine. The disclosure provides personalized cancer vaccine compositions and kits containing personalized cancer vaccine compositions.

Predicting whether a first neoantigen or a second neoantigen of a patient has a stronger binding affinity for a HLA class I complex of the patient uses artificial intelligence, statistical modeling, or a combination thereof. Examples of artificial intelligence that are useful in the disclosed methods include machine learning, e.g. artificial neural networks and support vector machines, and evolutionary computing, e.g. evolutionary algorithms. In some embodiments, the machine learning can include deep learning, e.g. deep artificial neural networks. In some embodiments, the estimating step includes statistical modeling, e.g. stochastic models or position specific soring models (PSSM). Stochastic models useful with the present methods include Markov models, e.g. hidden Markov models and Baum-Welch algorithms.

The predicting step includes estimating the binding affinity of two or more neoantigens to one or more HLA complexes of the patient. Such estimating includes artificial intelligence, statistical models, or a combination thereof. After two or more such HLA-neoantigen binding affinities are estimated, the estimated HLA-neoantigen binding affinities are compared in order to predict which neoantigen will have the strongest binding affinity for a HLA complex of the patient. In some embodiments, the predicting step includes estimating the binding affinity of two or more neoantigens to one or more HLA complexes of the patient, such as two or more, three or more, four or more, five or more, or six. In some embodiments, the predicting step includes estimating the stability of the MHC-neoantigen peptide complex, or the peptide affinity for binding to the MHC, of two or more neoantigens to one or more HLA complexes of the patient, such as two or more, three or more, four or more, five or more, or six. The HLA class I complexes of the patient can be determined from the HLA class I genotype of the patient according to methods well known in the art.

In some embodiments, the HLA complex is a HLA class I complex. In some embodiments, the HLA genotype is a HLA class I genotype.

The artificial intelligence and statistical models described above are based on training data, e.g. the presence of binding, the absence of binding, the strength of binding, or a combination thereof of antigens to major histocompatibility complex (MHC) class I complexes. In some cases, the MHC class I complex is a human leukocyte antigen (HLA) class I complex. In some cases, the MHC class I complex is that of a non-human animal, e.g. a rat or a mouse. Examples of experimental data useful with the present methods includes mass spectrometry data, crystal structure data, in silico modeling of antigen-HLA binding, in silico modeling of the three-dimensional structure of an antigen, in silico modeling of the three-dimensional structure of a HLA class I complex, and data of the kinetics of dissociation of an antigen-HLA complex for example, in response with challenge to increasing urea concentration.

In some embodiments, the method further includes identifying a first and a second neoantigen in the patient by obtaining genome data about the patient, wherein genome data includes one or more of genome, exome, transcriptome data from a normal cell and a cancer cell of the patient.

In some embodiments, the method further includes determining a HLA genotype of the patient. In some cases, the HLA genotype is a HLA class I genotype. In some cases, the HLA class I genotype is selected from: the HLA-A genotype, the HLA-B genotype, the HLA-C genotype, or a combination thereof.

The present disclosure provides personalized cancer vaccine compositions that include a particle comprising a material and a neoantigen, wherein the neoantigen is encapsulated by the material. In some cases, the personalized cancer vaccine includes a first and second particle, the first particle contains a first neoantigen (or multiple copies thereof) that is absent from the second particle, and the second particle contains a second neoantigen (or multiple copies thereof) that is absent from the first particle. In some cases, each particle only contains a single neoantigen, or multiple copies of that antigen. In some cases, a particle is substantially spherical has a diameter in the range of 11 micrometers ±20%, ±10%, ±5%, ±2%, or ±1%. In some cases, the particle is sized so that an antigen presenting cell can uptake one, and only one, particle.

The present disclosure provides methods of treating a cancer patient comprising administering a personalized cancer vaccine as described herein to a patient. The patient is in need of, or will be in need of, such treatment due to having cancer.

The present disclosure provides kits including personalized cancer vaccine as described herein and a label including instructions for administering the personalized cancer vaccine to the patient.

The present disclosure provides a method of treating triple negative breast cancer for tumors that do not produce the checkpoint inhibitor PD-L1.

These and other objects, advantages, and features of the invention will become apparent to those persons skilled in the art upon reading the details of the formulations and methods of treatment as more fully described below.

### BRIEF DESCRIPTION OF THE FIGURES

FIG. 1 is a schematic representation of processing steps used in connection with the invention. Human triple negative cancer cells (4T1 cell line) were subject to RNA sequencing and compared to RNA sequencing data from normal mouse breast tissue. 4T1 tumor cells showed over expression of Survivin tumor neoantigen QP19. The 4T1 cells were found to produce essentially the same levels of PD-L1 found in normal tissue.

FIG. 2 is a tabular presentation of expression data shown in FIG. 1. RNA sequencing of normal mouse breast tissue and 4T1 tumor tissue showed overexpression of Survivin protein tumor neoantigen QP19 on tumor cells (Fragments Per Kilobase Per Million Mapped Reads (FPKM) shown on the Y-Axis). The 4T1 cells were also found to not produce PD-L1 (very low levels by FPKM).

FIG. 3 is a graph showing the surviving mice in the treatment group had fewer tumors than the mice in the control group. The tenth mouse in the treatment group (not shown) died before tumor weight or ELISPOT measurements could be made

FIG. 4 is a graph wherein the ELISPOT assay shows that untreated mice did not mount a killer T-cell attack against the QP19 triple negative breast cancer tumor neoantigen after tumor injection.

FIG. 5 is a graph wherein the. ELISPOT data for the treated group shows a stronger level of T-cell attack against QP19 tumor neoantigen than seen in the untreated group. The surviving mouse in the treated group with tumor had a below average response to the vaccine.

### DETAILED DESCRIPTION OF THE INVENTION

Before the present composition, formulation and method of manufacture and use and treatment are described, it is to be understood that this invention is not limited to particular embodiment described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

A vaccine is a biological preparation intended to improve a recipient's immunity to a particular disease. A vaccine typically contains an agent that resembles a disease-causing microorganism, and is often made from weakened or killed forms of the microbe or its toxins. The agent stimulates the body's immune system to recognize the agent as foreign, destroy it, and “recognize” it, so that the immune system can more easily recognize and destroy any of these microorganisms that it later encounters. Vaccines can be prophylactic (e.g. to prevent or ameliorate the effects of a future infection by any natural or “wild” pathogen), or therapeutic (e.g. vaccines against cancer are also being investigated).

A human leukocyte antigen (HLA) complex is a human major histocompatibility complex (MHC). HLA complexes include HLA class I complexes and HLA class II complexes. HLA-A, HLA-B, and HLA-C are three types of human MHC class I complexes coded for by the HLA-A, HLA-B, and HLA-C loci, respectively.

A human leukocyte antigen (HLA) genome is the group of genes encoding the HLA complexes. The HLA genome includes the HLA class I genome and the HLA class II genome.

Programmed death-ligand 1 (PD-L1) is a protein that in humans is encoded by the CD274 gene. Programmed death-ligand 1 (PD-L1) is a 40 kDa type 1 transmembrane protein that has been speculated to play a major role in suppressing the adaptive arm of immune system during particular events such as pregnancy, tissue allografts, autoimmune disease and other disease states such as hepatitis. Normally the adaptive immune system reacts to antigens that are associated with immune system activation by exogenous or endogenous danger signals. In turn, clonal expansion of antigen-specific CD8+ T cells and/or CD4+ helper cells is propagated. The binding of PD-L1 to the inhibitory checkpoint molecule PD-1 transmits an inhibitory signal based on interaction with phosphatases (SHP-1 or SHP-2) via Immunoreceptor Tyrosine-Based Switch Motif (ITSM) motif. This reduces the proliferation of antigen-specific T-cells in lymph nodes, while simultaneously reducing apoptosis in regulatory T cells (anti-inflammatory, suppressive T cells)—further mediated by a lower regulation of the gene Bcl-2

The term “antigen” as used herein includes meanings known in the art, and means a molecule or portion of a molecule, frequently for the purposes of the present invention a polypeptide molecule (amino acid sequence), that can react with a recognition site on an antibody or T cell receptor. The term “antigen” also includes a molecule or a portion of a molecule that can, either by itself or in conjunction with an adjuvant or carrier, elicit an immune response (also called an “immunogen”).

The term “neoantigen” as used herein includes meanings known in the art, and means an antigen present on the surface of cancer cells but absent from the surface of normal cells of a patient. A neoantigen is at least about 8 amino acids in length, and not more than about 15 to 22 amino acids in length. A T cell receptor recognizes a more complex structure than antibodies, and requires both a major histocompatibility antigen binding pocket and an antigenic peptide to be present. The binding affinity of T cell receptors to epitopes is lower than that of antibodies to epitopes, and will usually be at least about 10-4 M, more usually at least about 10-5 M.

The term “antigen presenting cell” or APC may generally refer to a mammalian cell having a surface HLA class I or HLA class II molecule in which an antigen is presented. Unless otherwise indicated, for the purposes of the present invention an antigen presenting cell is a “professional” antigen presenting cell that can activate or prime T cells, including naïve T cells. A professional APC usually express both HLA Class I and HLA Class II molecules, and are very efficient at internalizing antigen, either by phagocytosis or by receptor-mediated endocytosis, and then displaying the antigen or a fragment thereof bound to the appropriate HLA molecule on their cell surface. Synthesis of additional co-stimulatory molecules is a defining feature of professional APCs. Of these APCs, dendritic cells (DCs) have the broadest range of antigen presentation, and are the most important T cell activators. Macrophages, B cells and certain activated epithelial cells are also professional APCs.

The term “ome data” refers to data about the genome, exome, transcriptome, or combination thereof of a patient.

The expression “enhanced immune response” or similar term means that the immune response is elevated, improved or enhanced to the benefit of the host relative to the prior immune response status, for example, a native status before the administration of an immunogenic composition of the invention.

The terms “cell-mediated immunity” and “cell-mediated immune response” are meant to refer to the immunological defense provided by lymphocytes, such as that defense provided by T cell lymphocytes when they come into close proximity to a target cell. A cell-mediated immune response normally includes lymphocyte proliferation. When “lymphocyte proliferation” is measured, the ability of lymphocytes to proliferate in response to a specific antigen is measured. Lymphocyte proliferation is meant to refer to T-helper cell or cytotoxic T-lymphocyte (CTL) cell proliferation.

The term “immunogenic amount” refers to an amount of antigenic compound sufficient to stimulate an enhanced immune response, when administered with a subject immunogenic composition, as compared with the immune response elicited by the antigen in the absence of the microsphere formulation.

The terms “treatment”, “treating”, “treat” and the like are used herein to generally refer to obtaining a desired pharmacologic and/or physiologic effect such as an enhanced immune response. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a subject, particularly a mammalian subject, more particularly a human, and includes: (a) preventing the disease or symptom from occurring in a subject which may be predisposed to the disease or symptom but has not yet been diagnosed as having it; (b) inhibiting the disease symptom, e.g., arresting its development; or relieving the disease symptom, i.e., causing regression of the disease or symptom (c) reduction of a level of a product produced by the infectious agent of a disease (e.g., a toxin, an antigen, and the like); and (d) reducing an undesired physiological response to the infectious agent of a disease (e.g., fever, tissue edema, and the like).

The “specificity” of an antibody or T cell receptor refers to the ability of the variable region to bind with high affinity to an antigen. The portion of the antigen bound by the immune receptor is referred to as an epitope, and an epitope is that portion of the antigen which is sufficient for high affinity binding. An individual antigen typically contains multiple epitopes, although there are instances in which an antigen contains a single epitope.

### Invention in General

Methods and compositions related to personalized cancer vaccines are disclosed. The disclosure provides methods of making a personalized cancer vaccine that includes predicting whether a first neoantigen or a second neoantigen of a particular individual patient has a stronger binding affinity for a human leukocyte antigen (HLA) complex of the patient and creating a particle containing the neoantigen with the stronger predicted binding affinity. Such a predicting step includes artificial intelligence, statistical modeling, or a combination thereof. Such a particle is created by encapsulating the neoantigen with the stronger predicted binding affinity for the HLA complex of the patient in a material. Placing the antigen in a particular sized particle is referred to here as Size Exclusion Antigen Presentation Control, (SEAPAC). The disclosure also provides methods of treating the patient using such a personalized cancer vaccine. The disclosure provides personalized cancer vaccine compositions and kits containing personalized cancer vaccine compositions.

In some embodiments, the method further includes identifying a first and a second neoantigen in the patient by obtaining ome data about the patient, wherein ome data includes one or more of genome, exome, transcriptome data from a normal cell and a cancer cell of the patient.

In some embodiments, the method further includes determining a HLA genotype of the patient. In some cases, the HLA genotype is a HLA class I genotype. In some cases, the HLA class I genotype is selected from: the HLA-A genotype, the HLA-B genotype, the HLA-C genotype, or a combination thereof.

### Predicting HLA-Neoantigen Binding Affinity

As described above, the step of predicting whether a first neoantigen or a second neoantigen of a patient has a stronger binding affinity for a HLA complex of the patient includes artificial intelligence, statistical modeling, or a combination thereof. Such a step is performed based on training data and the HLA genotype of the patient.

Artificial intelligence includes computational methods that attempt to predict an outcome of situation, e.g. the potential binding of a neoantigen to an HLA complex, based known outcomes from similar situations. As used herein, the known outcomes from similar situations are referred to as training data. As an example, training data might include whether particular neoantigens were found to bind, or to not bind, to a particular HLA complex, e.g. HLA-A*0201. As an example, such neoantigens might vary based on the first and last amino acid, e.g. AMFPNAPYL, AMFPNAPYP, and RMFPNAPYL. Thus, such training data can be used to predict whether a neoantigen with a different but similar amino acid sequence, e.g. RMFPNAPYP, will bind to HLA-A*0201. As used herein, the data set where it is unknown whether, or how strongly, a particular neoantigens will bind to a particular HLA complexes is referred to as test data. Robust data sets are generated and expanded by predicting novel neoantigen candidates that are validated in-vitro in cell culture media. A high-throughput assay can be created by binding fluorophores to the candidate neoantigen peptides in order to visualize a successful antigen presentation event. This is performed using adjuvanted microspheres each containing a single fluorophore labeled sequence. This visual event can be digitized by a microscope equipped with a light source with an appropriate wavelength to trigger a fluorescence event from the fluorophore bound to the neoantigen peptide. Cells for analysis of this kind using microscopy need to be placed in multi-well plates. These plates can employ metallic reflecting background material to enhance the fluorescence event from peptide bound fluorophores to enhance the ability to visualize the movement of fluorophore labeled peptides within the cell. It is also possible to visualize an antigen presentation event by pre-treating the cells in culture with flourophor labeled peptide antigen causing the MHC receptors on the surface of the antigen presenting cells to become saturated with labeled peptide. Then, microspheres with unlabeled peptide can be introduced into culture and light microscopy can be used to observe displacement of the labeled peptide from the surface of the cell by unlabeled peptide as antigen presentation occurs of the unlabeled peptide. This latter approach has the advantage that the same formulation intended for patient treatment can be used in the in-vitro assay as fluorophore labels do not have be incorporated into the peptides loaded into the microspheres.

This and other techniques can allow the movement of peptides from within the cell to the surface of the cell, indicating that a presentation event associated with neoantigen peptide-MHC binding has occurred.

This visual signal can be processed through various methods (e.g. convolutional neural networks) in order to deliver a score for HLA binding or other intracellular events associated with that neoantigen peptide, these other events include proteolytic cleavage and transportation of antigen via the TAP protein. The addition of infrared fluorophores to peptides for visualization does not interfere significantly with the peptide-MHC binding event as we have shown using in-vitro peptide-MHC binding affinity assays demonstrating that attaching a near-infrared window fluorophores (Zhu et al., PNAS Jan. 31, 2017, vol. 114, no. 5, Pages 962-967) to the pan-DR binding epitope (PADRE) peptide (AKFVAAWTLKAAA) maintains a physiologically appropriate MHC-peptide binding event (Table 1). Data validating MHC binding predictions can be generated through neural network enhanced analysis as described above creating valuable feedback into neural network models to better predict peptide-MHC binding properties for arbitrary neoantigen peptides. Antigen presentation is a necessary but not sufficient step to produce T-cell expansion in response to a peptide neoantigen. The ELISpot assay demonstrates the extent to which a specific neoantigen peptide produces a T cell expansion event after antigen presentation. This assay is performed by adding a peptide neoantigen being evaluated to a well of PBMCs on a ELISpot plate designed to cause a cell to change color if interferon gamma is released in response to that peptide neoantigen being presented and processed by an APC resulting in a T cell expansion event. By conducting ELISpot tests on the peripheral blood of the patient, neoantigen prediction algorithms can further benefit from this feedback which correlates to neoantigen processing and the physiological response (T cell expansion).

| | | PADRE + Flourophore | PADRE |
|---|---|---|---|
| | | Mean Value | Mean Value |
| DRB3*0202 | Kd(nM) | 61 | 50 |

As shown in the data above through the use of a peptide affinity binding assay, peptide-MHC binding affinity between PADRE and DRB3*0202 is shown to be essentially unchanged before and after the addition of an infrared fluorophore ligand.

Artificial intelligence is distinguished from hard-coded methods in that hard-coded methods include parameters are explicitly specified by a human. In contrast, artificial intelligence methods use computational methods to adjust various parameters of the model, without explicit human direction, in order to accurately reflect the training data in a way that would allow the best possible predictions for test data. In a hard-coded version of the example described above, a human would explicitly examine whether the AMFPNAPYL, AMFPNAPYP, and RMFPNAPYL neoantigens bind to the HLA-A*0201 neoantigen, and explicitly decide whether the first amino acid, the last amino acid, or both influenced the HLA-neoantigen binding. The hard-coded method would then predict the outcome of the test data, e.g. RMFPNAPYP and HLA-A*0201, based on the explicit human hypotheses.

In contrast, in an artificial intelligence method, a human would provide the computational system with training data, but the computation system would modify the parameters of the model based on the training data in order to obtain the best predictions for test data. The addition of more training data as it becomes available allows the computational system to continue learning and improve predictions through various implementations and architectures.

One example of artificial intelligence is machine learning. Machine learning can be divided into various categories based on different aspects of the process, e.g. supervised versus unsupervised learning. In supervised learning, the computational system attempts to optimize the model by adjusting parameters that are identified by a human as potentially influencing the outcome. In regard to the example above, a human might identify the first and last amino acid as potentially relevant to binding to the HLA-A*0201 complex, and the computational system would consider those variables in the model. In unsupervised learning, the computational system is not explicitly instructed which parameters are potentially important to the outcome, and therefore identifies potentially relevant parameters based on the training data. As an example, the computational system might hypothesize that the relationship between the first two amino acids, e.g. AM versus RM, are relevant to HLA-neoantigen binding.

An example of a machine learning technique useful with the present methods is artificial neural networks (ANNs). ANNs are so named due to being inspired by biological brains. ANNs include multiple so-called layers including one input layer, one or more hidden layers, and one output layer, wherein each layer has various nodes. Starting at the input layer, each node is connected to one or more nodes at the next layer, and each connection has a weighting coefficient. In the analogy to a biological brain, each node is a neuron. Training data is parsed into individual parameters, which are then assigned to corresponding input nodes. Based on the value of each node and the weighting factor between nodes, the values so-called propagate from the input layer through the hidden layer to the output layer. In the example above, the input layer would be the amino acid sequence of the neoantigens and then properties of the HLA complex. In the example above, the output layer would be whether the neoantigen binds to the HLA complex, or how strongly such binding occurs. In order to improve the accuracy of predicting HLA-neoantigen binding, the weighting factors of each connection between nodes can be varied in order to best fit the training data. In ANNs wherein there are two or more hidden layers, the ANN is referred to as a deep ANN. Machine learning techniques extend past neural networks to clustering, random forest, etc.

Support vector machines (SVMs) are another example of a machine learning technique useful with the present methods. SVMs are supervised learning methods useful for classification and regression analysis. Whereas ANNs can be used to predict the magnitude of an outcome, e.g. the strength of HLA-neoantigen binding, SVMs are used to predict one of several discrete outcomes, e.g. whether the neoantigen will bind or not to the HLA complex.

Another example of artificial intelligence useful with the present method is evolutionary algorithms. Evolutionary algorithms borrow concepts from biological evolution in order to improve the ability of training data to predict the outcome of test data. Evolutionary algorithms involve random or pseudo-random changes in various parameters, i.e. similar to mutations in biological systems, followed by assessment of whether the new parameters more accurately model the training data, i.e. analogous to the biological concept of evolutionary fitness.

The prediction of HLA-neoantigen binding can also involve statistical models, e.g. position specific scoring models (PSSMs) and Markov models. Statistical models are distinguished from artificial intelligence in that artificial intelligence involves the adjustment of various parameters over multiple iterations, wherein the similarity between the model and the training data is assessed after each iteration. In contrast, statistical models do not involve such multiple iterations, but instead involve executing pre-defined algorithms in order to predict the outcome of the test data. Like with artificial intelligence, predicting the strength of HLA-neoantigen binding with statistical models involves the use of generated training data.

Using a PSSM in the present methods involves deciding on a length of neoantigen to consider, e.g. a neoantigen of 8 amino acids, 9 amino acids, etc. Once a length of neoantigen is decided, each amino acid is labeled as a distinct position, i.e. the position with which it interacts with the HLA complex. Next, a matrix of numerical values is constructed, wherein the row can be the amino acid position and the column can be the identity of the amino acid, e.g. histidine (H), lysine (K), etc. The numerical values in each cell, i.e. each combination of a particular position and amino acid, can reflect the relative importance to HLA-neoantigen binding. As an example, if the training data shows or suggests that a histidine amino acid at the 4^{th} position strongly increases binding affinity, then the 4-histidine cell can be assigned a relatively large numerical value, e.g. +18. In contrast, if a lysine at the 4^{th} position strongly disfavors binding affinity based on the training data, then it can be assigned a lower value, e.g. −9. If the identity of the amino acid at a particular position does not appear to meaningfully influence binding affinity, it can be assigned a value with a relatively small absolute value, e.g. −2 for mildly disfavor binding or +1 for mildly favor binding. The relative weights of all the values in the matrix can be adjusted or determined by the training data. In order to predict the binding strength for a neoantigen in the test data, the values corresponding to the amino acids in each position can be summed and compared to sum of known HLA-neoantigen complexes. Since the amino acid sequence of the neoantigen is varied while the HLA complex is kept constant, the PSSM is most useful when the test data's HLA complex is identical to, or highly similar to, the HLA complex used to construct the PSSM.

A Markov model is a type of statistical model used to model randomly changing systems. A hidden Markov model is a type of Markov model, and is the simplest representation of a dynamic Bayesian network. Another type of Markov model is the Markov chain. The Baum-Welch algorithm is one manner of finding the unknown parameters in a hidden Markov model. Each of the Markov models described herein are useful with the present methods.

The training data used in each of the above described manners of predicting HLA-neoantigen binding can originate from various sources, and can be of various types. Such training data includes a plurality of entries, wherein each entry includes (i) the amino acid sequence or three-dimensional chemical structure of an antigen, or a combination thereof; (ii) the amino acid sequence or three-dimensional chemical structure or identity of an HLA complex, or a combination thereof; and (iii) a description of the HLA-antigen binding, e.g. presence of binding, absence of binding, or strength of binding.

As is understood by those skilled in the art, the HLA genotype of a patient refers to the particular alleles of the genes that code for the HLA complexes that are carried by the patient. HLA complexes relevant to the present methods include HLA class I complexes, which include HLA-A, HLA-B, and HLA-C complexes, and HLA class II complexes, which include HLA-DP, HLA-DM, HLA-DO, HLA-DQ, and HLA-DR complexes. HLA class II complexes are also relevant to the present methods.

Since patients typically carry two copies of the HLA genes, i.e. one from each parent, a patient will typically have two different alleles for HLA-A. In some cases, a patient will inherit the same HLA-A allele from both parents, and so the patient will only have one HLA-A allele. Thus, most patients will have six HLA complex I alleles and six HLA complex I complexes: two HLA-A, two HLA-B, and two HLA-C. One example of the identity of an HLA-A allele and complex is HLA-A*0201.

As such, estimating the strength of HLA-neoantigen binding as described herein is performed using a particular neoantigen and a particular HLA complex, which corresponds to a particular HLA allele. In some cases, the method includes estimating the binding affinity of two neoantigens for one particular HLA complex. In some cases, the method includes estimating the binding affinity of two neoantigens for two or more particular HLA complexes, such as three or more HLA complexes, four or more HLA complexes, five or more HLA complexes, or six HLA complexes.

Hence, predicting whether a first or second neoantigen will have a stronger binding affinity for an HLA complex can include estimating the HLA-neoantigen binding strength for multiple particular HLA complexes.

In some cases, the training data includes amino acid sequence data, e.g. of the neoantigen. In some cases, the training data includes amino acid sequence data of the HLA complex. In some cases, the training data includes the three-dimensional chemical structure of the neoantigen, the HLA complex, the HLA-neoantigen complex, or a combination thereof. Such three-dimensional chemical structure data can be obtained from crystal structure analyses, in silico modeling of the relevant chemical structures, or any other manner known in the art Amino acid data can be obtained in any manner known in the art, e.g. mass spectrometry. In some cases, the presence, absence, or strength of HLA-neoantigen binding is obtained from crystal structure analysis, mass spectrometry, in silico modeling, kinetics of dissociation analysis, or any combination thereof. In some cases, the training data describes the presence or absence of HLA-neoantigen binding. In some cases, the training data describes the strength of the HLA-neoantigen binding. Neoantigen processing is not solely dependent on the neoantigen peptide sequence. Flanking amino acid sequences can affect the way neoantigens are processes and presented. Training data relative to these flanking region is important. Neoantigens arise from more than just somatic mutations that are missense, i.e. causing an amino acid change not seen in the germline. Other ways in which neoantigens can arise includes frameshift mutations, alternative splicing events, translated non-coding regions, and neo-reading frames. Next-generation sequencing for DNA and RNA can uncover the presence of these expressed sequences.

### Identifying Neoantigens

Neoantigens can be identified by comparing the genome, exome, transcriptome, or a combination thereof of one or more normal cells to the genome, exome, transcriptome, or a combination thereof of one or more cancer cells. As described above, the term “neoantigen” as used herein includes meanings known in the art, and means an antigen present on the surface of cancer cells but absent from the surface of normal cells of a patient. As used herein, the term “ome data” refers to data about the genome, exome, transcriptome, or combination thereof of a patient.

Samples of tissue from which normal cells and cancer cells can be obtained include fresh biopsies, frozen or otherwise preserved tissue or cell samples, circulating cancer cells, exosomes, various bodily fluids, e.g. blood, etc.

After obtaining the relevant cells, suitable manners of obtaining ome data include nucleic acid sequencing, and particularly NGS methods operating on DNA (e.g., Illumina sequencing, ion torrent sequencing, 454 pyrosequencing, nanopore sequencing, etc.), RNA sequencing (e.g., RNAseq, reverse transcription-based sequencing, etc.), and protein sequencing or mass spectroscopy-based sequencing (e.g., SRM, MRM, CRM, etc.). Sequencing specifications to retrieve the human exome and/or genome from extracted DNA can include various steps to improve capture and downstream analysis such as using PCR free library. For RNA-Seq, preparation steps involving different capture methods such as Poly-A, Ribosomal depletion, etc. can be utilized to effectively capture areas of interest. Likewise, the computational analysis of the sequence data may be performed in numerous manners. In most preferred methods, however, analysis is performed in silico by location-guided synchronous alignment of tumor and normal samples as, for example, using BAM files and BAM servers, disclosed in US Patent Publications 2012/0059670 and 2012/0066001, which are herein incorporated by reference for manners of obtaining ome data and identifying neoantigens. Additional bioinformatics formats used by software or artificial intelligence algorithms may also include FASTQ, VCF, (G)VCF, FASTQC, FASTA, etc. Such analysis advantageously reduces false positive neoepitopes and significantly. The genetic sequencing of the genome of a cancer may be performed by techniques readily known to one skilled in the art or by using standard procedures, as described, for example, in U.S. Patent Publication No. 2011/0293637, which is herein incorporated by reference for manners of obtaining ome data.

Neoantigens can be identified by comparing ome data from normal cells to ome data of cancer cells, e.g. by filtering by at least one of mutation type, transcription strength, translation strength, and a priori known molecular variations. As an example, the high-affinity binder has an affinity to the at least one HLA class I sub-type or the at least one HLA class II sub-type of less than 150 nM, and/or the HLA genotype of the patient is determined in silico using a de Bruijn graph. An example of such comparison of ome data is described in US Patent Publication 2017/0028044, which is incorporated by reference for manners of identifying neoantigens.

Mutations in cancer cells may be identified by considering the type (e.g., deletion, insertion, transversion, transition, translocation) and impact of the mutation (e.g., non-sense, missense, frame shift, etc.), which may serve as a first content filter through which silent and other non-relevant (e.g., non-expressed) mutations are eliminated.

In addition, since a neoantigen includes several amino acids, e.g. 8, 9, 10, 11, a single mutation in a cancer cell can produce several neoantigens. Framed in another way, the change in amino acid can appear at any position throughout the neoantigen, e.g. the first amino acid, the second amino acid, the third amino acid the last amino acid. Thus, after a neoantigen is identified based on a change in amino acid, additional neoantigens that contain the same changed amino acid can also be identified. Thus, a single mutation can result in multiple neoantigens which can be assessed for their binding affinity for a HLA complex, increasing the probability that a strong binding affinity will be found.

If the HLA complex is a HLA class I complex, a typical neoantigen length will be about 8-11 amino acids, while the typical neoantigen length for presentation via a HLA class II complex will have a length of about 13-17 amino acids. As will be readily appreciated, since the position of the changed amino acid in the neoepitope may be other than central, the actual amino acid sequence of the neoantigen and the actual topology of the neoantigen may vary considerably.

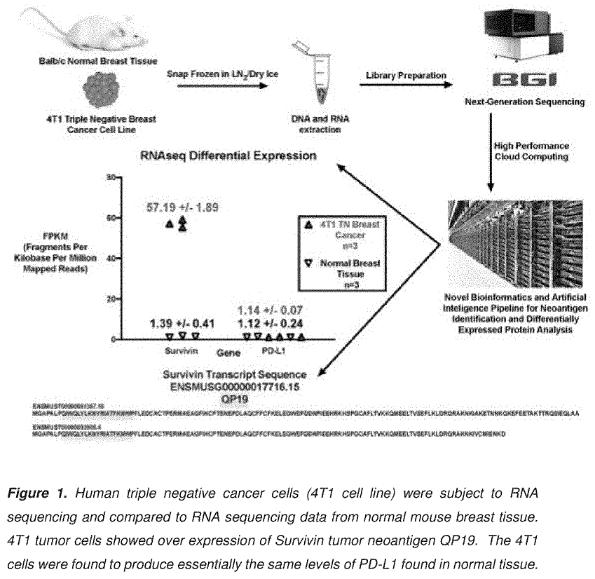

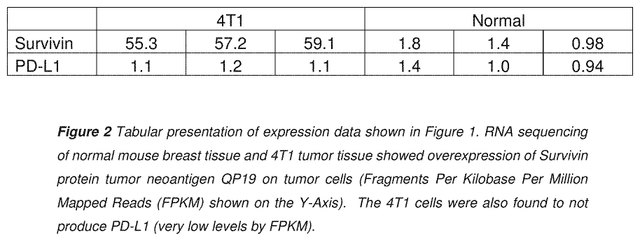

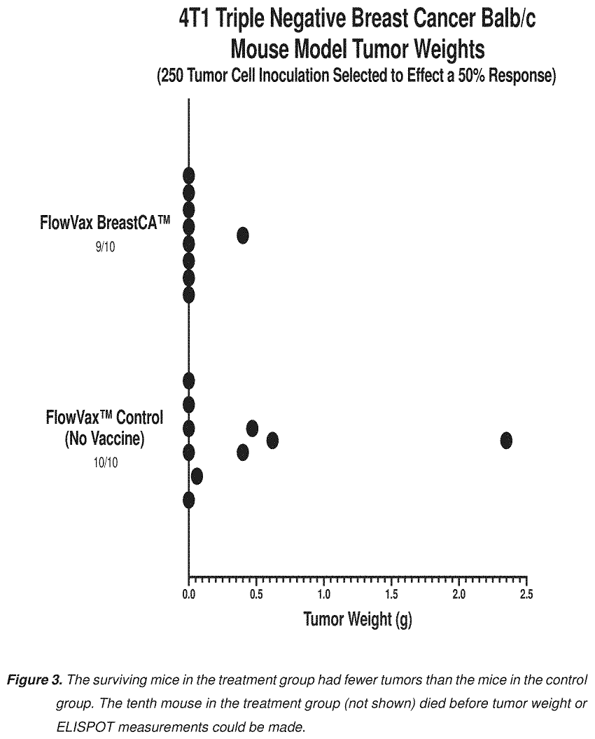

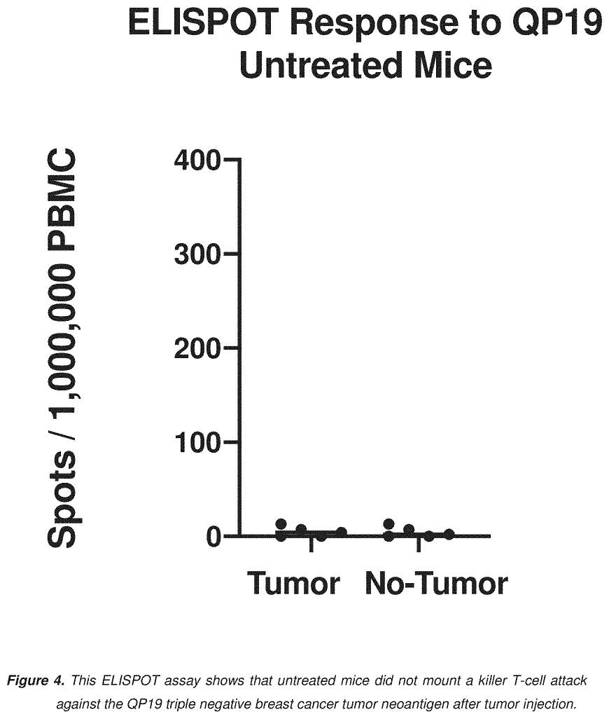

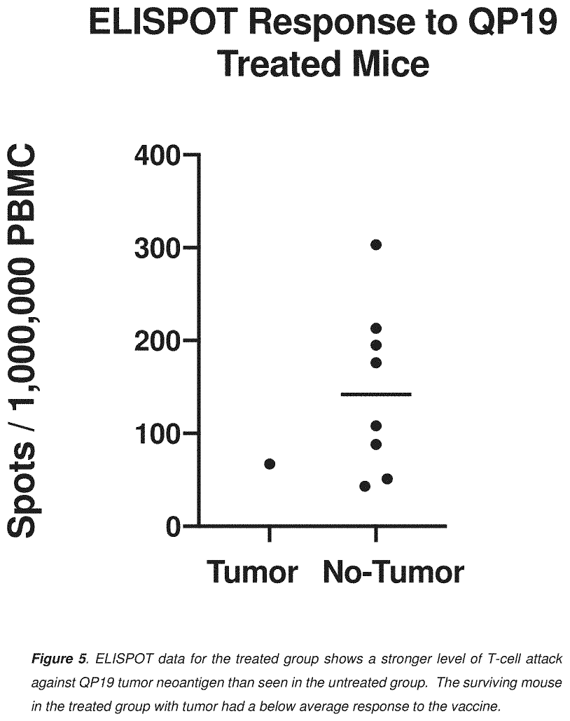

FIG. 1 schematically shows the process of neo-antigen identification in a mouse using the 4T1 triple negative breast cancer tumor model in BALB/c mice. This study evaluated the tumor suppressive effect of FlowVax BreastCA™ microspheres loaded with peptide neoantigen QP19, determined by RNA sequencing to be present on triple negative breast cancer cells but not on normal breast tissue (see FIGS. 1 and 2). A 4T1 dose of 250 cells, predicted in our previous studies to produce tumor in 50% of the control mice, was delivered by injection into the breast tissue of two groups of 10 mice each, with one group serving as control and the other group designed to evaluate efficacy of FlowVax BreastCA™. Preliminary data shows that the surviving mice receiving FlowVax BreastCA™ 14 days before tumor injection, and a second dose with tumor injection, had fewer tumors than the mice in the control group (FIG. 3). The mice in the treated group showed a stronger immune response against tumor neoantigen QP19 than untreated mice (FIGS. 4 and 5).

This study also showed that the 4T1 tumor in these mice did not elaborate the checkpoint inhibitor PD-L1 which is present in less than half of triple negative breast cancer tumors. This is especially relevant because the currently FDA approved specific treatment for triple negative breast cancer involves the use of antibodies directed against PD-L1 or PD-1. These treatments have been shown to be effective when PD-L1 is being produced by the tumor. In fact, atezolizumab, marketed by Genentech as TECENTRIQ, is co-marketed with an immunohistochemistry assay to detect the presence of PD-L1 in a triple negative tumor sample so as to guide the treating physician to use TECENTRIQ if PD-L1 is present in the tumor sample. The current invention is specifically intended to be used to treat triple negative breast cancer when PD-L1 is not being produced by the tumor cells.

### Determining HLA Genotype

As described above, a patient has a plurality of HLA complexes, wherein each HLA complex corresponds to a particular allele of one of the plurality of genes that code for HLA complexes. As an example, patients typically have six HLA class I alleles and complexes: two HLA-A, two HLA-B, and two HLA-C. Thus, determining the HLA genotype of a patient means determining the identity of one or more of the alleles or complexes in a patient. In some cases, the determining includes determining the two or more alleles in a patient, such as three or more alleles, four or more alleles, five or more alleles, or six or more alleles.

Any method known in the art for determining the HLA genotype of a patient can be used, such as sequencing the whole genome of the patient and identifying one or more allele that codes for an HLA complex. Methods known in the art include those of US Patent Application 2010/008691, which is incorporated by reference for methods of determining the HLA genotype of a patient.

### Creating Particles

The step of creating a particle of the present invention involves encapsulating the neoantigen with the stronger predicted binding affinity for a HLA complex of the patient in a material.

The neoantigen to be encapsulated in the particle can be obtained by any suitable method, e.g. chemically synthesizing a neoantigen. Several methods of chemically synthesizing a neoantigen are known in the art. As a neoantigen contains a plurality of amino acids, methods of synthesizing peptides are relevant to the synthesis of neoantigens. Solution phase peptide synthesis can be used to construct neoantigens of moderate size or, for the chemical construction of neoantigens, solid phase synthesis can be employed. Atherton et al. (1981) Hoppe Seylers Z. Physiol. Chem. 362:833-839. Proteolytic enzymes can also be utilized to couple amino acids to produce neoantigens. Kullmann (1987) Enzymatic Peptide Synthesis, CRC Press, Inc. Alternatively, the neoantigen can be obtained by using the biochemical machinery of a cell, or by isolation from a biological source. Recombinant DNA techniques can be employed for the production of neoantigens. Hames et al. (1987) Transcription and Translation: A Practical Approach, IRL Press. Neoantigens can also be isolated using standard techniques such as affinity chromatography.

The material of a particle can be any of a variety of compositions, e.g. a polymer. In some cases, the polymer is a biocompatible polymer. Examples of biocompatible polymers useful in the present invention include hydroxyaliphatic carboxylic acids, either homo- or copolymers, such as poly(lactic acid), poly(glycolic acid), poly(dl-lactide/glycolid), poly(ethylene glycol); polysaccharides, e.g. lectins, glycosaminoglycans, e.g. chitosan; celluloses, acrylate polymers, and the like. In some cases the biocompatible polymer is poly(lactic-co-glycolic acid) (PLGA), polycaprolactone, polyglycolide, polylactic acid, or poly-3-hydroxybutyrate. In some cases, the particle includes two or more different materials.

The particle can be created by any suitable method, e.g. by mixing the neoantigen with the material and extruding the mixture from a device, as described in U.S. Pat. No. 6,116,516, which is incorporated herein by reference for a method of making a particle. The particle can also be created according to methods described in U.S. Pat. Nos. 9,408,906, and 10,172,936 both of which are incorporated herein by reference for a method of making a particle, e.g. with a particular size, and when used with the technology described here provide for a new type of vaccine referred to as Size Exclusion Antigen Presentation Control, (SEAPAC).

In some embodiments the particle is a microsphere. The microsphere can be substantially spherical. The microsphere can have a range of diameters, e.g., a diameter within the range of 1 micron (i.e. micrometer) to 100 microns. The particle can have a diameter in the range of 11 micrometers ±20%, ±10%, ±5%, ±2%, or ±1%. In some cases the microsphere has a diameter between 2 microns and 50 microns, between 2 microns and 35 microns, between 2 microns and 20 microns, between 2 microns and 15 microns, between 2 microns and 10 microns, between 4 microns and 35 microns, between 4 microns and 20 microns, between 4 microns and 15 microns, between 4 microns and 10 microns, between 8 microns and 20 microns, between 8 microns and 15 microns, between 10 microns and 20 microns, or between 10 microns and 15 microns. The particle may have a diameter of about 4 microns, about 6 microns, about 8 microns, about 10 microns, about 12 microns, about 14 microns, about 16 microns, about 18 microns, about 20 microns, about 22 microns, about 24 microns, about 26 microns, about 28 microns, or about 30 microns.

In addition, the present disclosure provides groups of particles. In some cases, particles in a group can all have the same size, or all the particles in a group can have sizes within the same range. In other cases, the particles in a group can have different sizes, e.g. at least one particle in the group has a size that is different than the size of at least one other particle. In some embodiments every particle in the group has a diameter in the range of 11 micrometers ±20%, ±10%, ±5%, ±2%, or ±1%.

In some cases, all the particles in a group can encapsulate the same peptide species, i.e. multiple copies of the same peptide. As used herein, a peptide species is a peptide with a particular amino acid sequence such that peptides from different peptides species will have different amino acid sequences. In some cases, the particles in a group can encapsulate different peptide species, e.g. a first particle encapsulates a first peptide (or multiple identical copies of that peptide) species that is not encapsulated by a second particle, and the second particle encapsulates a second peptide species (or multiple identical copies of that peptide) that is not encapsulated by the first particle. As such, a plurality of groups of particles contain a plurality of peptide species, such as at least 2, at least 3, at least 4, at least 5, at least 10, at least 20, at least 30, at least 40, at least 50, or more. In addition, particles from multiple groups can be combined in order to form a new group of particles.

In one embodiment, a first and second groups of particles are created that encapsulate a first and second peptide species, respectively. The first and second groups of particles are then combined such that the resulting combination of particles is a personalized cancer vaccine containing the first and second peptide species. Such a combination of particles can also be made using three, four, five, six, or more groups of particles encapsulating third, fourth, fifth, sixth, etc. peptide species, respectively, such that the personalized cancer vaccine contains three, four, five, six, or more peptide species. A personalized cancer vaccine can also contain only a single peptide species. Furthermore, the plurality of particles in a personalized cancer vaccine can contain particles with any combination of sizes, materials, and peptide species.

The size of a particle can be designed such that an antigen presenting cell, such as a dendritic cell, can consume only a single particle. There is evidence that presentation of a plurality of epitopes by a single APC may result in immunodominance of a single epitope, which is undesirable in situations where overall responsiveness to the plurality of epitopes is desirable. For example, see Rodriguez, et al, “Immunodominance in Virus-Induced CD8+ T-Cell Responses Is Dramatically Modified by DNA Immunization and Is Regulated by Gamma Interferon” Journal of Virology, 76(9):4251-4259 (May 2002) and Yu et al., “Consistent Patterns in the Development and Immunodominance of Human Immunodeficiency Virus Type I (HIV-1)-Specific CD8+ T-Cell Responses following Acute HIV-1 Infection” Journal of Virology, 76(17):8690-9701 (September 2002), both incorporated herein by reference. Hence, designing particles with a size such that an antigen present cell can only consume a single particle will allow a population of antigen-presenting cells to present plurality of antigen species. A given antigen presenting cell will take up and present only a limited number of antigen species, e.g., less than 5, less than 3, usually a single species.

The optimum size of particle to achieve the desired result may vary depending on the charge of the peptide that is being presented, e.g., a positively charged peptide may be more readily ingested by an antigen presenting cell than a neutral or negatively charged peptide. In some embodiments each peptide is individually optimized for a microsphere size that achieves exclusive uptake, and thus a formulation of a plurality of microsphere/peptide combinations may be heterogenous in size, although the size for a peptide species will be narrowly defined.

The optimal size of a particle may depend upon the type of antigen presenting cell that consumes the particle. The three major classes of antigen presenting cells are dendritic cells, macrophages, and B cells. However, the size of a particle may be optimized for any type of antigen presenting cell, including without limitation immature dendritic cells, monocytes, mature myeloid dendritic cells, etc. In some embodiments the particle size is optimized for the type of antigen presenting cell that consumes the particle. In other embodiments the particle size is not optimized for the type of antigen presenting cell that consumes the particle.

The three major classes of antigen presenting cells are dendritic cells (DCs), macrophages, and B cells, but dendritic cells are considerably more potent on a cell-to-cell basis and are the only antigen presenting cells that activate naïve T cells. DC precursors migrate from bone marrow and circulate in the blood to specific sites in the body, where they mature. This trafficking is directed by expression of chemokine receptors and adhesion molecules. Upon exposure to antigen and activation signals, the DCs are activated, and leave tissues to migrate via the afferent lymphatics to the T cell rich paracortex of the draining lymph nodes. The activated DCs then secrete chemokines and cytokines involved in T cell homing and activation, and present processed antigen to T cells. The groups of particles of the invention provide information on how to best present processed antigens to T cells to obtain a desired immune response.

DCs mature by upregulating costimulatory molecules (CD40, CD80 and CD86), and migrate to T cell areas of organized lymphoid tissues where they activate naive T cells and induce effector immune responses. In the absence of such inflammatory or infectious signals, however, DCs present self-antigens in secondary lymphoid tissues for the induction and maintenance of self-tolerance. Dendritic cells include myeloid dendritic cells and plasmacytoid dendritic cells.

For purposes of the invention, e.g. determining the uptake of particles of any formulation including a vaccine formulations by APC, any one of the classes of APC may be used, including without limitation immature DC, monocytes, mature myeloid DC, mature pDC, etc. For example see Foged et al (2005) International Journal of Pharmaceutics 298(2): 315-322; Reece et al. (2001) Immunology and Cell Biology 79:255-263; Tel et al. (2010) J. Immunol. 184:4276-4283, each herein specifically incorporated by reference.

In some cases, the particle is sized so that a dendritic cell will uptake one, and only one, particle, which for a human system is generally a particle that is substantially spherical, and has a diameter in the range of 11 micrometers ±20%, ±10%, ±5%, ±2%, or ±1%.

### Compositions

The present disclosure provides personalized cancer vaccine compositions that include a particle comprising a material and a neoantigen, wherein the neoantigen is encapsulated by the material.

In some embodiments, the neoantigen is embedded in material, e.g. by mixing the neoantigen and the material prior to formation of a particle. In other embodiments, the neoantigen is coupled to the surface of the particle. The surface may be optionally textured to simulate, to a degree, the surface of an infectious bacteria, virus or other pathogen.

The material of a particle can be any of a variety of compositions, e.g. a polymer. In some cases, the polymer is a biocompatible polymer. Examples of biocompatible polymers useful in the present invention include hydroxyaliphatic carboxylic acids, either homo- or copolymers, such as poly(lactic acid), poly(glycolic acid), poly(dl-lactide/glycolid), poly(ethylene glycol); polysaccharides, e.g. lectins, glycosaminoglycans, e.g. chitosan; celluloses, acrylate polymers, and the like. In some cases the biocompatible polymer is poly(lactic-co-glycolic acid) (PLGA), polycaprolactone, polyglycolide, polylactic acid, or poly-3-hydroxybutyrate. In some cases, the particle includes two or more different materials.

In some embodiments the particle is a microsphere. The microsphere can be substantially spherical. The microsphere can have a range of diameters, e.g., a diameter within the range of 1 micron (i.e. micrometer) to 100 microns. In some cases the microsphere has a diameter between 2 microns and 50 microns, between 2 microns and 35 microns, between 2 microns and 20 microns, between 2 microns and 15 microns, between 2 microns and 10 microns, between 4 microns and 35 microns, between 4 microns and 20 microns, between 4 microns and 15 microns, between 4 microns and 10 microns, between 8 microns and 20 microns, between 8 microns and 15 microns, between 10 microns and 20 microns, between 10 microns and 15 microns, or between 9 and 13 microns. The particle may have a diameter of about 4 microns, about 6 microns, about 8 microns, about 10 microns, about 11 microns, about 12 microns, about 14 microns, about 16 microns, about 18 microns, about 20 microns, about 22 microns, about 24 microns, about 26 microns, about 28 microns, or about 30 microns. The particle can have a diameter in the range of from 10 micrometers ±20% to 25 micrometers ±20%. The particle can have a diameter in the range of 11 micrometers ±20%, ±10%, ±5%, ±2%, or ±1%.

The particle size can be selected to (a) be sufficiently small that it is capable of uptake and processing by an antigen presenting cell; and (b) be sufficiently large that an APC will generally take up not more than one particle. The size of a particle can be designed such that an antigen presenting cell, such as a dendritic cell, can consume only a single particle. Hence, designing particles with a size such that an antigen present cell can only consume a single particle will allow a population of antigen-presenting cells to present plurality of neoantigens. A given antigen presenting cell will take up and present only a limited number of neoantigens, e.g., less than 5, less than 3, usually a single neoantigen. In some cases, the particle has a size such that a dendritic cell will take up only a single particle.

The optimum size for a particular peptide or class of peptides may be determined empirically by various methods. For example, two different peptides may be detectably labeled with two different fluorophores, and used to prepare particles of the invention. A mixture of the particles is provided to antigen presenting cells, which are then viewed by optical microscope, flow cytometry, etc. to determine if a single fluorophore or if multiple fluorophores are present in any single APC, where the size of particle that provides for exclusive uptake is chosen. Functional tests may also be performed, e.g. by providing particles with the cognate antigens for different T cell lines and determining if one or both lines are activated by an APC.

In order to determine the precise size which is desirable for the particles, various types of labeling can be used. In addition to the fluorophores referred to above, labeling can be performed with semiconductor nanocrystals which are generally referred to as quantum dots. The purpose of carrying out the experiment is to determine a size at which the antigen presenting cells such as the macrophage can consume only a single particle. The size would be too large if the macrophage cannot consume the particle. The size would be too small if the macrophage can consume more than one particle.

The optimum size of particle to achieve the desired result may vary depending on the charge of the neoantigen that is being presented, e.g., a positively charged neoantigen may be more readily ingested by an antigen presenting cell than a neutral or negatively charged neoantigen. In some embodiments each neoantigen is individually optimized for a microsphere size that achieves exclusive uptake, and thus a formulation of a plurality of particle/neoantigen combinations may be heterogenous in size, although the size for a neoantigen will be narrowly defined.

The optimal size of a particle may depend upon the type of antigen presenting cell that consumes the particle. The three major classes of antigen presenting cells are dendritic cells, macrophages, and B cells. However, the size of a particle may be optimized for any type of antigen presenting cell, including without limitation immature dendritic cells, monocytes, mature myeloid dendritic cells, etc. In some embodiments the particle size is optimized for the type of antigen presenting cell that consumes the particle. In other embodiments the particle size is not optimized for the type of antigen presenting cell that consumes the particle.

In some embodiments, the personalized cancer vaccine includes a first and second particle. Such particles may be heterogenous or homogenous in size, usually homogeneous, where the variability may be not more than 100% of the diameter, not more 50%, not more than 20%, not more than 10%, not more than 2%, etc. Particle sizes are may be about 8 microns in diameter, about 10 microns, about 12 microns about 14 microns, about 15 microns, about 16 microns, about 17 microns, about 18 microns, about 20 microns, not more than about 25 microns diameter.

In some cases, the personalized cancer vaccine includes a first and second particle, the first particle contains a first neoantigen that is absent from the second particle, and the second particle contains a second neoantigen that is absent from the first particle. In some cases, each particle only contains a single neoantigen.

In addition, the present disclosure provides groups of particles. In some cases, particles in a group can all have the same size, or all the particles in a group can have sizes within the same range. In other cases, the particles in a group can have different sizes, e.g. at least one particle in the group has a size that is different than the size of at least one other particle.

In some cases, all the particles in a group can include the same neoantigen. In some cases, the particles in a group can include different neoantigens, e.g. a first particle includes a first neoantigen that is not encapsulated by a second particle, and the second particle includes a second neoantigen that is not present in the first particle. As such, a plurality of particles in a group of particles can contain a plurality of neoantigens, such as at least 2, at least 3, at least 4, at least 5, at least 10, at least 20, at least 30, at least 40, at least 50, or more neoantigens. In addition, particles from multiple groups can be combined in order to form a new group of particles.

In some embodiments, a first and second groups of particles are created that include first and second groups of neoantigens, respectively. The first and second groups of particles are then combined such that the resulting combination of particles is a personalized cancer vaccine containing the first and second neoantigens. Such a combination of particles can also be made using three, four, five, six, or more groups of particles including third, fourth, fifth, sixth, etc. neoantigens, respectively, such that the personalized cancer vaccine contains three, four, five, six, or more neoantigens. A personalized cancer vaccine can also contain only a single neoantigens. Furthermore, the plurality of particles in a personalized cancer vaccine can contain particles with any combination of sizes, materials, and neoantigens.

In some cases, the personalized cancer vaccine further comprises one or more antibiotics to prevent growth of bacteria during production and storage of the vaccine. One skilled in the art would recognize that a variety of antibiotic compositions could be used with the present invention.

In some cases, the personalized cancer vaccine further comprises one or more preservatives, one or more stabilizers, or a combination thereof to help the vaccine to remain unchanged during storage of the vaccine. Several preservatives are available, including thiomersal, phenoxyethanol, and formaldehyde. Monosodium glutamate (MSG) and 2-phenoxyethanol are used as stabilizers in a few vaccines to help the vaccine remain unchanged when the vaccine is exposed to heat, light, acidity, or humidity. Phenoxyethanol is another preservative that can be combined with the personalized cancer vaccine. Thimerosal is a mercury-containing preservative that is added to vials of vaccine that contain more than one dose to prevent contamination and growth of potentially harmful bacteria. Thiomersal is more effective against bacteria, has better shelf life, and improves vaccine stability, potency, and safety, but in the U.S., the European Union, and a few other affluent countries, it is no longer used as a preservative in childhood vaccines, as a precautionary measure due to its mercury content. Although controversial claims have been made that thiomersal contributes to autism, no convincing scientific evidence supports these allegations.

In some cases, the personalized cancer vaccine further comprises one or more pharmaceutically acceptable vehicles such as saline, Ringer's solution, dextrose solution, and the like. Generally, the personalized cancer vaccines are formulated for administration by injection or inhalation, e.g., intraperitoneally, intravenously, subcutaneously, intramuscularly, etc. Accordingly, these compositions are preferably combined with pharmaceutically acceptable vehicles such as saline, Ringer's solution, dextrose solution, and the like.

In some cases, the personalized cancer vaccine further comprises a pharmaceutically acceptable excipient. As is well known in the art, a pharmaceutically acceptable excipient is a relatively inert substance that facilitates administration of a pharmacologically effective substance. For example, an excipient can provide form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and non-parenteral drug delivery are set forth in Remington's Pharmaceutical Sciences 19th Ed. Mack Publishing (1995). The following excipients are commonly present in compositions to generate an immune response such as vaccine preparations. Aluminum salts or gels are added as adjuvants. Adjuvants are added to promote an earlier, more potent response, and more persistent immune response to the vaccine; they allow for a lower vaccine dosage. Antibiotics are added to some vaccines to prevent the growth of bacteria during production and storage of the vaccine. Egg protein is present in influenza and yellow fever vaccines as they are prepared using chicken eggs. Other proteins may be present. Formaldehyde is used to inactivate bacterial products for toxoid vaccines. Formaldehyde is also used to kill unwanted viruses and bacteria that might contaminate the vaccine during production. Monosodium glutamate (MSG) and 2-phenoxyethanol are used as stabilizers in a few vaccines to help the vaccine remain unchanged when the vaccine is exposed to heat, light, acidity, or humidity. Thimerosal is a mercury-containing preservative that is added to vials of vaccine that contain more than one dose to prevent contamination and growth of potentially harmful bacteria.

### Methods of Treatment

The invention also includes methods of treating a cancer patient comprising administering a personalized cancer vaccine as described herein to a patient. The patient is in need of, or will be in need of, such treatment due to having cancer

The personalized cancer vaccine can be administered to the patient by methods including without limitation orally, intravenously, intraperitoneally, intramuscularly, intrathecally, subcutaneously, topically, cutaneously, transdermally, rectally, vaginally, optically, by the mouth, by the nose, or any other route. In some cases, personalized cancer vaccine can be formulated for administration orally, intravenously, intraperitoneally, intramuscularly, intrathecally, subcutaneously, topically, cutaneously, transdermally, rectally, vaginally, parenterally, naso-pharyngeal, pulmonarily, opitically, by the mouth, by the nose, or by any other route.

Parenteral routes of administration include but are not limited to electrical (iontophoresis) or direct injection such as direct injection into a central venous line, intravenous, intramuscular, intraperitoneal, intradermal, or subcutaneous injection. Compositions suitable for parenteral administration include, but are not limited, to pharmaceutically acceptable sterile isotonic solutions. Such solutions include, but are not limited to, saline and phosphate buffered saline for injection of the compositions.

Naso-pharyngeal and pulmonary routes of administration include, but are not limited to, inhalation, transbronchial and trans alveolar routes. The invention includes compositions suitable for administration by inhalation including, but not limited to, various types of aerosols for inhalation, as well as powder forms for delivery systems. Devices suitable for administration by inhalation of include, but are not limited to, atomizers and vaporizers. Atomizers and vaporizers filled with the powders are among a variety of devices suitable for use in inhalation delivery of powders.

The effective amount and method of administration of a particular formulation can vary based on the individual patient and other factors evident to one skilled in the art. The absolute amount given to each patient depends on pharmacological properties such as bioavailability, clearance rate and route of administration.

The dose, timing, etc. of administration of the personalized cancer vaccine can be adjusted based on the patient's medical history, response to one or more previous administrations of the personalized cancer vaccine, or other clinical parameters.

In some cases, the personalized cancer vaccine can be co-administered to a patient with one or more additional compositions. As used herein, co-administered refers to both combining the personalized cancer vaccine with one or more additional compositions and administering the combination to the patient, and also to administering the personalized cancer vaccine and the one or more additional compositions separately, e.g., the administrations of the personalized cancer vaccine and the additional compositions are separated by a certain amount of space, time, or both.

In some embodiments, the personalized cancer vaccine can be co-administered with one or more immunogenic agents. As used herein, immuno-stimulatory agent is used interchangeably with immuno-stimulatory agent. As with all immunogenic compositions, the immunologically effective amounts and method of administration of the particular formulation can vary based on the individual, what condition is to be treated and other factors evident to one skilled in the art. Factors to be considered include the immunogenicity, route of administration and the number of doses to be administered. Such factors are known in the art and it is well within the skill of oncologist to make such determinations without undue experimentation. A suitable dosage range is one that provides the desired modulation of immune response to cancer cells based on the neoantigen. Generally, a dosage range may be, for example, from about any of the following, referencing the amount of peptide in a dose exclusive of carrier: 0.01 to 100 μg, 0.01 to 50 μg, 0.01 to 25 μg, 0.01 to 10 μg, 1 to 500 μg, 100 to 400 μg, 200 to 300 μg, 1 to 100 μg, 100 to 200 μg, 300 to 400 μg, 400 to 500 μg. Alternatively, the doses can be about any of the following: 0.1 μg, 0.25 μg, 0.5 μg, 1.0 μg, 2.0 μg, 5.0 μg, 10 μg, 25 μg, 50 μg, 75 μg, 100 μg. Accordingly, dose ranges can be those with a lower limit about any of the following: 0.1 μg, 0.25 μg, 0.5 μg and 1.0 μg; and with an upper limit of about any of the following: 250 μg, 500 μg and 1000 μg. The absolute amount given to each patient depends on pharmacological properties such as bioavailability, clearance rate and route of administration.

In some embodiments, personalized cancer vaccine can be co-administered with one or more pharmaceutically acceptable excipient. As is well known in the art, a pharmaceutically acceptable excipient is a relatively inert substance that facilitates administration of a pharmacologically effective substance. For example, an excipient can provide form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington's Pharmaceutical Sciences 19th Ed. Mack Publishing (1995).

In some embodiments, the personalized cancer vaccine can be co-administered with one or more adjuvant. The immunogenic composition may contain an amount of an adjuvant sufficient to potentiate the immune response to the immunogen. Adjuvants are known in the art and include, but are not limited to, oil-in-water emulsions, water-in oil emulsions, alum (aluminum salts), liposomes and microparticles including but not limited to, polystyrene, starch, polyphosphazene and polylactide/polyglycosides. Other suitable adjuvants also include, but are not limited to, MF59, DETOX™ (Ribi), squalene mixtures (SAF-1), muramyl peptide, saponin derivatives, mycobacterium cell wall preparations, monophosphoryl lipid A, mycolic acid derivatives, nonionic block copolymer surfactants, Quil A, cholera toxin B subunit, polyphosphazene and derivatives, and immunostimulating complexes (ISCOMs) such as those described by Takahashi et al. (1990) Nature 344:873-875, as well as, lipid-based adjuvants and others described herein. For veterinary use and for production of antibodies in animals, mitogenic components of Freund's adjuvant (both complete and incomplete) can be used.

In some embodiments, the personalized cancer vaccine can be co-administered with one or more immunomodulatory facilitators. Thus, the invention provides compositions comprising plurality of microspheres of defined size comprising distinct antigen species and an immunomodulatory facilitator. As used herein, the term “immunomodulatory facilitator” refers to molecules which support and/or enhance immunomodulatory activity Immunomodulatory facilitators include, but are not limited to, co-stimulatory molecules (such as cytokines, chemokines, targeting protein ligand, trans-activating factors, peptides, and peptides comprising a modified amino acid) and adjuvants (such as alum, lipid emulsions, and polylactide/polyglycolide microparticles).

In some cases, the personalized cancer vaccine can be co-administered with one or more checkpoint inhibitors in order to increase immune function. The checkpoint inhibitor can include without limitation ipilmumab, nivolumab, pembrolizumab, atezolizumab, avelumab, and durvalumab.

In some cases, the method of treatment involves use of a delivery system.

### Delivery Systems

The methods of producing suitable devices for injection, topical application, atomizers and vaporizers are known in the art and will not be described in detail.

The above-mentioned compositions and methods of administration are meant to describe but not limit the methods of administering the compositions of the invention. The methods of producing the various compositions and devices are within the ability of one skilled in the art and are not described in detail here.

There are several new delivery systems in development to make vaccine delivery more efficient. Methods include liposomes and ISCOM (immune stimulating complex). Other vaccine delivery technologies have resulted in oral vaccines. A polio vaccine was developed and tested by volunteer vaccinations with no formal training; the results were positive in that the ease of the vaccines increased dramatically. With an oral vaccine, there is no risk of blood contamination. Oral vaccines are likely to be solid which have proven to be more stable and less likely to freeze; this stability reduces the need for a “cold chain”: the resources required to keep vaccines within a restricted temperature range from the manufacturing stage to the point of administration, which, in turn, will decrease costs of vaccines.

A microneedle approach may be used, where the microneedle, which is “pointed projections fabricated into arrays that can create vaccine delivery pathways through the skin”. Microneedles (MN), as used herein, refers to an array comprising a plurality of micro-projections, generally ranging from about 25 to about 2000 μm in length, which are attached to a base support. An array may comprise 10², 10³, 10⁴, 10⁵ or more microneedles, and may range in area from about 0.1 cm² to about 100 cm². Application of MN arrays to biological membranes creates transport pathways of micron dimensions, which readily permit transport of macromolecules such as large polypeptides. The microneedle array may be formulated as a transdermal drug delivery patch. MN arrays can alternatively be integrated within an applicator device which, upon activation, can deliver the MN array into the skin surface, or the MN arrays can be applied to the skin and the device then activated to push the MN through the skin.

### Kits

The disclosure also provides kits including a personalized cancer vaccine as described herein and a label comprising instructions for administering the personalized cancer vaccine to the patient.

It is to be understood that this invention is not limited to the particular methodology, protocols, peptides, animal species or genera, constructs, and reagents described, as such may, of course, vary. It is also to be understood that the terminology used herein is for the purpose of describing particular embodiments only, and is not intended to limit the scope of the present invention, which will be limited only by the appended claims.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the reagents, cells, constructs, and methodologies that are described in the publications, and which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. A method of making a personalized cancer vaccine for a patient, comprising:
a) identifying a first and a second neoantigen in the patient;
b) determining the human leukocyte antigen (HLA) genotype of the patient;
c) predicted whether the first neoantigen or the second neoantigen has a stronger binding affinity for a HLA complex of the patient based on training data and the HLA genotype of the patient; and
d) creating a particle by encapsulating in a material the neoantigen predicted to have the stronger binding affinity for the HLA complex of the patient.

2. The method of claim 1 where the tumor is a triple negative breast cancer tumor that does not produce programmed death-ligand 1 (PD-L1) above a level of 1.5 fragments per kilobase per million mapped reads.

3. The method of claim 1, wherein the predicting comprises using a methodology selected from the group consisting of artificial intelligence methodology, machine learning, artificial neural network methodology, and deep artificial neural network methodology.

4. The method of claim 3, wherein the machine learning comprises support vector machines.

5. The method of claim 3, wherein the artificial intelligence comprises an evolutionary algorithm, and wherein the predicting comprises statistical modeling.

6. The method of claim 5, wherein the statistical modeling is position specific scoring modeling.

7. The method of claim 5, wherein the statistical modeling is a Markov model.

8. The method of claim 7, wherein the Markov model comprises a hidden Markov model.

9. The method of claim 8, wherein the predicting further comprises a Baum Welch algorithm.

10. The method of claim 1, wherein the training data is selected from the group consisting of amino acid sequence data and three-dimensional chemical structure data.

11. The method of claim 10, wherein the three-dimensional chemical structure data comprises any one of: crystal structure data, in silico modeling of the binding of the HLA complex with the first neoantigen, in silico modeling of the binding of the HLA complex with the second neoantigen, or a combination thereof.

12. The method of claim 10, where the training data comprises visualization of peptide antigen presentation using fluorophore labeled peptides and light microscopy.

13. The method of the claim 12, where the fluorophores are placed on peptides loaded within microspheres incubated with antigen presenting cells.

14. The method of claim 13, where the fluorophores are placed on peptides incubated with antigen presenting cells.

15. The method of claim 14, where the fluorophores are placed on peptides incubated with antigen presenting so as to saturate mhc receptors on the surface of antigen presenting cells.

16. The method of claim 10, where the training data comprises ELISpot data from peripheral blood.

17. The method of claim 10, wherein the HLA genotype is an HLA class I genotype and the HLA complex is a HLA class I complex; and
wherein the identifying step is selected from the group consisting of (a) obtaining genome data from a normal cell of the patient; (b) obtaining exome data from a normal cell of the patient; (c) obtaining transcriptome data from a normal cell of the patient; (d) obtaining genome data from a cancer cell of the patient; (e) obtaining exome data from a cancer cell of the patient; and (f) obtaining transcriptome data from a cancer cell of the patient.

18. The method of claim 1, wherein the material is a biocompatible polymer selected from the group consisting of poly(lactic-co-glycolic acid) (PLGA), polycaprolactone, polyglycolide, polylactic acid, poly-3-hydroxybutyrate;
wherein the particle is substantially spherical; and
has a diameter such that only a single particle can be consumed by an antigen presenting cell.

19. The method of claim 18, wherein the particle has a diameter in the range of 11 micrometers ±10%; and
wherein the neoantigen consists of between eight to twenty amino acids.

20. A method of making a personalized cancer vaccine, comprising the steps of:
a) obtaining a plurality of nucleotide sequences from a tumor cell of a patient;
b) obtaining a plurality of nucleotide sequences from a normal cell of the same patient;
c) interpreting the nucleotide sequences from the tumor cell and the normal cell to obtain a plurality of amino acid sequences for both the tumor cell and the normal cell;
d) identifying a plurality of tumor amino acid sequences which are amino acid sequences present in the tumor cell and absent from the normal cell;
e) determining the human leukocyte antigen (HLA) genotype of the patient;
f) predicted which of the plurality of tumor amino acid sequences has a stronger binding affinity for a HLA complex of the patient based on training data and the HLA genotype of the patient; and
creating a particle by encapsulating in a material a tumor amino acid sequence predicted to have strong binding affinity for a HLA complex of the patient relative to other tumor sequences.
